(19) United States
(12) Patent Application Publication
(10) Pub. No.: US 2006/0224047 A1
(43) Pub. Date: Oct. 5, 2006

(54) Sleepiness prediction apparatus and sleepiness prediction method

(75) Inventors: Takuji Suzuki, Kanagawa (JP); Kenichi Kameyama, Kanagawa (JP)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: Kabushiki Kaisha Toshiba, Tokyo (JP)

(21) Appl. No.: 11/232,988

(22) Filed: Sep. 23, 2005

(30) Foreign Application Priority Data
Mar. 30, 2005 (JP) 2005-100160

(51) Int. Cl. A61B 5/00; A61M 16/00; A61B 5/103

(52) U.S. Cl. 600/300; 128/204.23; 600/595; 128/920

(57) ABSTRACT

Since the sleepiness degree is changed by a daytime activity, a time and a sleep state, a sleepiness prediction apparatus in view of these is provided. The sleepiness prediction apparatus 10 includes a sensor head 14 mounted on a fingertip, and a main body 12 of a wrist watch type, measures a sleep state relevant value relevant to a sleep state of a test subject, measures a daytime activity relevant value relevant to a daytime activity of the test subject, calculates, based on the sleep state relevant value and the daytime activity relevant value, an accumulated sleepiness degree predicted to be accumulated by a sleep history of the test subject and a daytime activity, calculates a biological rhythm sleepiness degree based on a biological rhythm changing according to a time, and calculates a comprehensive sleepiness degree corresponding to a time based on the accumulated sleepiness degree and the biological rhythm sleepiness degree.

### CROSS-REFERENCE TO RELATED APPLICATIONS

This application is based upon and claims the benefit of priority from the prior Japanese Patent Application No. 2005-100160, filed on 30 Mar. 2005; the entire contents of which are incorporated herein by reference.

### TECHNICAL FIELD

The present invention relates to a sleepiness prediction apparatus which predicts daytime sleepiness based on information of a sleep state measured in daily life and notifies it to the user, and a sleepiness prediction method.

### BACKGROUND OF THE INVENTION

A driver's doze during driving of a railroad, an automobile or the like becomes a subject of discussion, and accident prevention by detecting the doze is demanded. For the detection of the doze, there are a method of measuring an increase in the number of blinks by using a camera, a method of measuring by a change of skin potential, a method by a frequency analysis of a variation in heartbeat, a method by a response speed of handle operation, a method by a change in conversation voice (see JP-A-2001-14599) and the like.

On the other hand, at present, the measurement of a sleep state is mainly performed using a detector called a sleep polysomnograph. The sleep polygraph is a device for simultaneously measuring biological signals of brain waves, ocular movement, myoelectricity, heart electricity and the like. From the pattern of change of these biological signals, a judgment of sleep depth (1 to 4) as a sleep state, REM sleep and the like is made by visual inspection or automatic analysis. The device is large and expensive, and is basically used only in a hospital specializing in sleep disorder.

The judgment of the sleep state is mainly based on the change of the brain waves due to the change of brain activities during the sleep. It is said that the brain is rested in the non-REM sleep, and memory is organized in the REM sleep. The brain waves, ocular movement and the like are changed by such active state. The sleep state is confirmed by grasping this.

On the other hand, it is known that during the sleep, an autonomic nervous system is also changed in accordance with the sleep state. The autonomic nervous system is also changed correspondingly to the change of the brain activity. Besides, for the purpose of the rest of a body, the autonomic activities such as breathing and beat are performed as efficiently as possible. It is said that the state of the sleep can be grasped by grasping such change. The autonomic nervous activity can be relatively easily measured from the heart electricity, pulse wave and the like. By this, the sleep state can be easily confirmed also in daily life. In patent document 1 (JP-A-2002-291710) or the like, sleep state estimation based on, especially, the frequency component of heartbeat variability in the autonomic nervous activities is performed. In patent document 2 (JP-A-2002-34955), a distinction is made among awakening, REM sleep, and non-REM sleep, as sleep states, by combination of the heartbeat and body movement.

As stated above, as the detection of a doze, the detecting techniques in which attention is paid to only the physiological phenomenon due to the sleepiness have been developed. However, in these methods, since the sleepy state is measured, even if the detection is performed, it is expected that the driving has already been in a dangerous state.

Originally, it is necessary that the occurrence of future sleepiness is notified before becoming sleepy, and an action such as driving is changed. However, in the conventional method, this is difficult.

The sleepiness is often dependent on the quality of previous day's sleep, and it is obvious that a time zone when the sleepiness occurs is after lunch. Thus, it is expected to be appropriate to predict the sleepiness from the sleep state on the previous night and on the night before the previous night, and the time.

Besides, the results obtained by the conventional sleep measurement method absolutely relate to the measurement of the depth of sleep, and the difference of the quality (non-REM sleep or REM sleep), and it has been difficult to judge whether, for example, at the time of uprising, the sleep up to that time has been eventually sufficient. For that purpose, since the past sleep, daytime activities and the like have influence, it is necessary to consider the accumulated influence of these.

Therefore, the invention relates to a sleepiness prediction apparatus which can predict sleepiness in daily life by a simple structure and a sleepiness prediction method.

### BRIEF SUMMARY OF THE INVENTION

According to embodiments of the invention, a sleepiness prediction apparatus includes a sleep state measurement processing part of measuring a sleep state relevant value relevant to a sleep state of a test subject, a daytime activity acquisition part of inputting or measuring a daytime activity relevant value relevant to a daytime activity of the test subject, and an accumulated sleepiness degree calculation processing part of calculating, based on the sleep state relevant value and the daytime activity relevant value, an accumulated sleepiness degree predicted to be accumulated by a sleep history of the test subject and the daytime activity.

In the invention, the accumulated sleepiness degree can be grasped based on the sleep history and the daytime activity up to now, and it becomes possible to perform the sleepiness prediction with high accuracy.

### BRIEF DESCRIPTION OF THE DRAWINGS

FIG. 1 is a structural view of a sleepiness prediction apparatus illustrating an embodiment of the invention.

FIG. 2 is a perspective view of the sleepiness prediction apparatus.

FIG. 3 is a perspective view of another structure of the sleepiness prediction apparatus.

FIG. 4 is a flowchart of a body movement and awakening judjment.

FIG. 5 is a flowchart of acquisition of autonomic nervous index values.

FIG. 6 is a view showing results of a frequency analysis.

FIG. 7 is a perspective view of a table of respective parameters corresponding to events.

FIG. 8 is a structural view of a sleepiness prediction apparatus in which an illumination sensor is added.

FIG. 9 is an explanatory view of a two-process model.

FIG. 10 is a view of a table of predicted sleepiness degrees in events.

FIG. 11 is a view showing an example of an alarm of a sleepiness degree.

FIG. 12 is a view showing input of daytime activities.

FIG. 13 is a view showing schedule data.

### DETAILED DESCRIPTION OF THE INVENTION

Hereinafter, a sleepiness prediction apparatus 10 of an embodiment of the invention will be described with reference to the drawings.

(1) Structure of the Sleepiness Prediction Apparatus 10

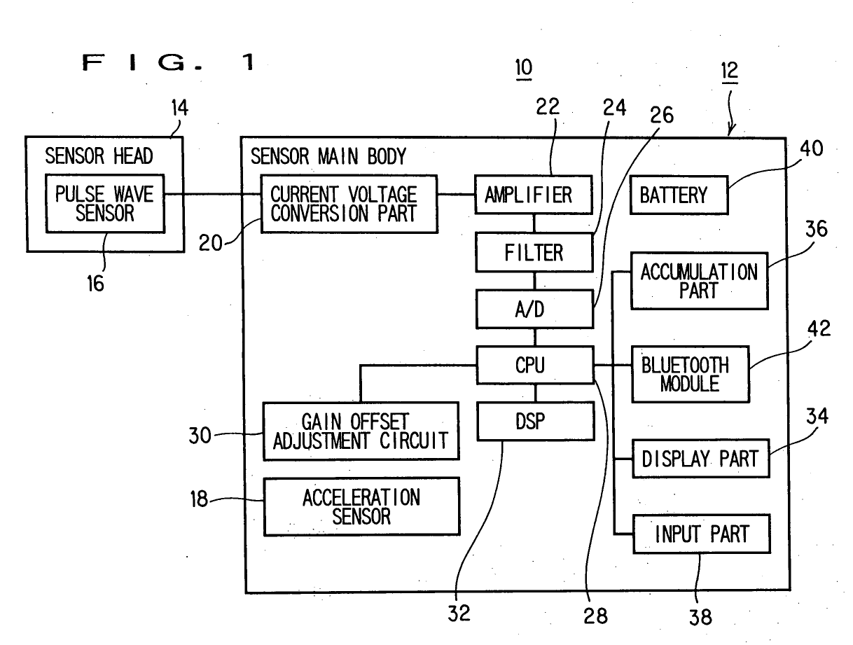

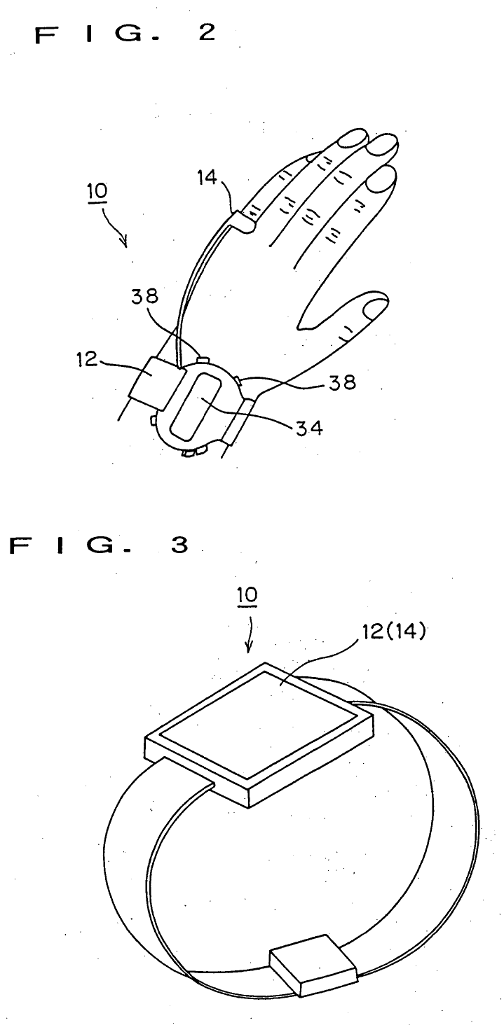

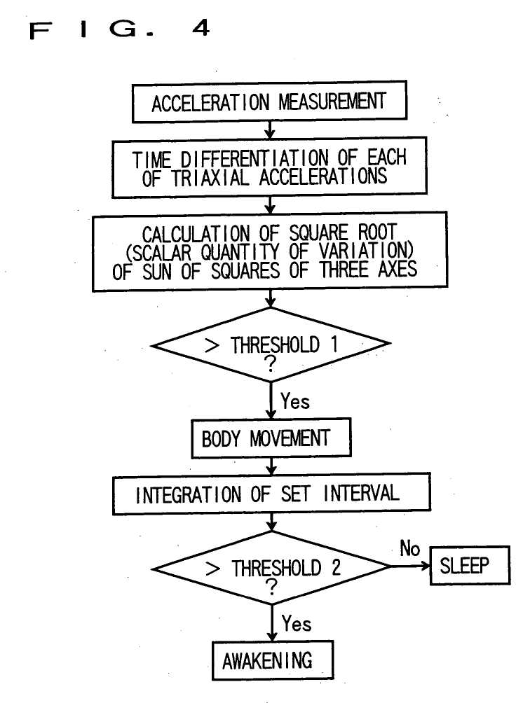

FIG. 1 is a block diagram showing a structural example of hardware of the sleepiness prediction apparatus 10 of the embodiment. Here, as shown in FIG. 2, a description will be made while using a wrist watch type as an example.

The sleepiness prediction apparatus 10 includes a sensor head 14 mounted on a fingertip, and a wrist watch type main body 12.

The sensor head 14 has a built-in pulse wave sensor 16 including a blue LED and a photodiode. The main body 12 has a built-in acceleration sensor 18. As the acceleration sensor 18, for example, a triaxial type one can measure acceleration of −2 G to 2 G.

The skin surface of the finger is irradiated by the blue LED of the pulse wave sensor 16, and a variation of a reflected wave changing by a blood flow change in a capillary vessel is grasped by the photodiode, so that the pulse wave is measured.

The output current from the photodiode is converted into a voltage by a current voltage conversion part 20 of the main body 12, and then, is amplified by an amplifier 22. The voltage is processed by a filter 24 including a high-pass filter (for example, its cutoff frequency is 0.1 Hz) for suppressing a large baseline drift and a low-pass filter (its cutoff frequency is 50 Hz) for cutting high frequency noise, and then, is A/D converted by a 10-bit A/D converter 26, and the data is inputted to a CPU 28.

Besides, after the gain and offset of analog output of the acceleration sensor 18 are adjusted by a gain offset adjustment circuit 30, similarly to the above, it is A/D converted by the 10-bit A/D converter, and then, is inputted to the CPU 28.

The CPU 28 performs respective signal processings described below while using a DSP 32, and displays a sleep state and sleep abnormality, as processing results, on a display part 34. Besides, the CPU 28 has also a clock function to measure a time.

Raw data after the A/D conversion, and data of a sleep state and the like after the processing are recorded and accumulated in an accumulation part 36. As the accumulation part 36, for example, a built-in flash memory is used. However, instead thereof, a slot for an external memory such as a flash memory card is incorporated, and the data may be accumulated in an external memory.

Besides, an input part 38 for the operation of the sleepiness prediction apparatus 10, a battery 40, and a Bluetooth module 42 for communicating with the outside are also incorporated.

(2) Operation Procedure of the Sleepiness Prediction Apparatus 10

A description will be given to a procedure in which in the sleepiness prediction apparatus 10, the sleep history and daytime activities are measured, and the sleepiness is predicted.

(2-1) Measurement of Sleep State

First, the sleep state is measured.

First, after the judgment of awakening/sleep, the sleep state (REM, non-REM [deep/right]) is judged. Here, an autonomic nervous activity is acquired from a pulse wave, and the sleep state is judged based thereon.

(2-1-1) Judgment of Awakening/Sleep

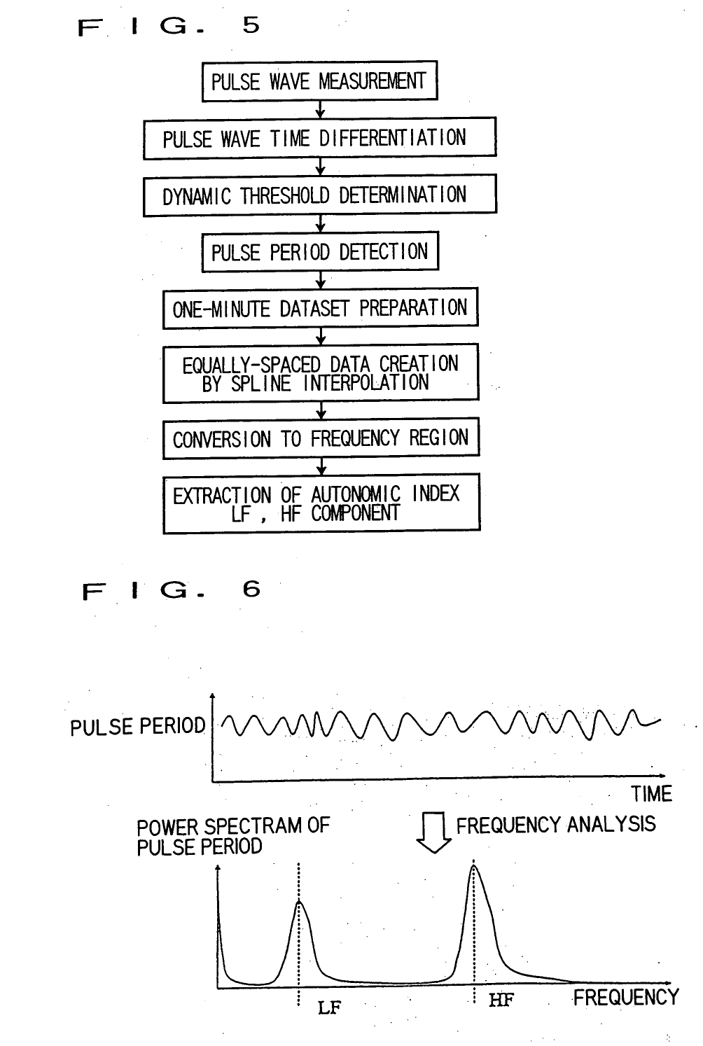

The amount of body movement is acquired from acceleration data, and a judgment of awakening/sleep is made. FIG. 4 shows a specific flowchart of the body movement and awakening judgment.

As variation amount of measured triaxial acceleration data, the time differentiation of each of them is performed. The square root of sum of squares of the time differential data is acquired, and the scalar quantity of the variation is obtained. This value is compared with a previously set threshold, and when it exceeds this, it is judged that the body movement occurs.

Next, in the case where the body movement occurs at a set frequency threshold or higher in a preceding interval, it is judged that waking has continued in the interval. When not exceeding the threshold, it is judged that sleep has continued in the interval.

(2-1-2) Acquisition of Autonomic Nervous Index Values

FIG. 5 shows a flow of acquisition processing of autonomic nervous index values from a pulse wave.

The pulse wave is subjected to the processing of the filter 24 and the A/D conversion, and then, is captured by the CPU 28, and the CPU 28 performs preprocessing of a waveform and detection of a pulse period.

As the preprocessing of the waveform, a signal processing for robusting with the body movement is performed. First, the time differentiation of the waveform is acquired, and the fluctuation of a DC component is removed. Thereafter, a dynamic threshold acquisition processing is performed. In the dynamic threshold acquisition processing, the maximum value and the minimum value of the pulse wave data in approximately one second before and after the processing point are acquired. Here, although the one second before and after the processing point is adopted, it may be replaced by the time interval of the pulse obtained just before.

Next, the acquisition processing of the pulse period is performed. The threshold obtained by the dynamic threshold acquisition processing is compared with the present and the last sampling data, and in the case where the sampling data increases across the threshold (that is, the last sampling data is smaller than the threshold, and the present data is larger than that), the pulse period is acquired. The pulse period is acquired from a different between the time when the data previously similarly exceeded the threshold and this latest time. As the time when the data exceeds the threshold, this latest time when it exceeded or the last time is used. After the pulse period data in which the influence of the body movement is reduced is acquired, a data set in a definite interval (for example, one minute) is created. When the data set is completed, the frequency analysis is performed, and the autonomic nervous index values LF and HF are acquired.

Since this data set is unequally-spaced data of the pulse period, an interpolation processing for creating equally-spaced data becomes necessary for the frequency analysis. The unequally-spaced pulse period data is interpolated and is resampled, and the equally-spaced data is created. For example, a cubic spline interpolation method is used, a predetermined number of points (for example, three points for each of front and back) are used, and the equally-spaced data between them is created.

When the equally-spaced data is created, this is frequency analyzed by using, for example, an FFT method. As the frequency analysis method, although any of the AR model, maximum entropy method, and wavelet may be used, in view of a realtime processing on a device, the FFT method of lightweight processing is used here.

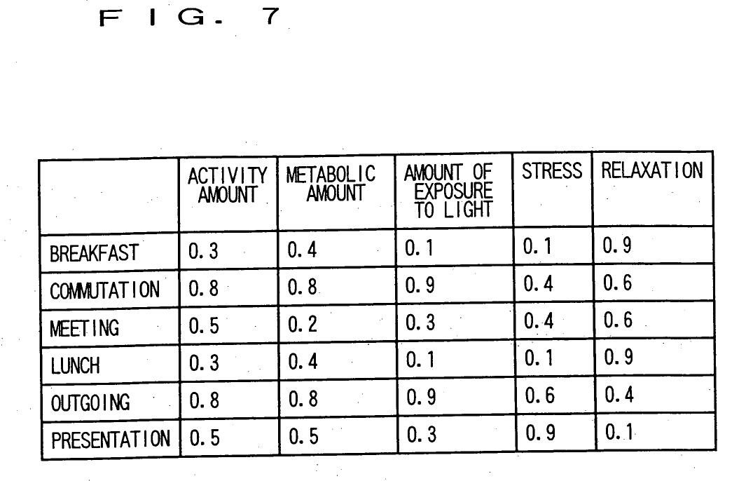

As a result of the frequency analysis, for example, as shown in FIG. 6, two peaks corresponding to LF and HF are seen. An average in a setting range with the maximum point of each frequency range as the center is made the power of each of LF and HF.

(2-2) Sleep State Relevant Values

Sleep state relevant values are calculated from the value of the acceleration sensor 18, the values of LF and HF, and the degree of fluctuation. The sleep state relevant values are a deep sleep time, a REM sleep time, the number of times of REM sleep, and a total sleep time.

First, a judgment of awakening or sleep is made by using the value of the acceleration sensor 18.

Next, in the case where HF has a threshold of 1 or higher, and LF/HF has a threshold of 2 or lower, it is judged that the sleep state is the non-REM deep sleep. On the contrary, in the case where HF has a threshold of 3 or lower, LF/HF has a threshold of 4 or higher, and the standard deviation of LF/HF in a predetermined time has a threshold of 5 or higher, it is judged that the sleep state is the REM sleep.

Based on the sleep state judged as stated above, the deep sleep time, the REM sleep time, the number of times of REM sleep, and the total sleep time are calculated, and are accumulated in the accumulation part 36 every night.

The deep sleep time is the total time of the deep sleeps occurring in the period of from bedtime to uprising, the REM sleep time is the total time of the REM sleeps in the period of from bedtime to uprising, the number of times of REM sleep is the number of times of REM sleep in the period of from bedtime to uprising, and the total sleep time is the time from bedtime to uprising.

(2-3) Daytime Activity Relevant Values

Next, daytime activity relevant values relevant to daytime activities are inputted. As the daytime activity relevant values, a daytime activity amount/metabolic amount, the amount of exposure to light, and a stress value are objects. Incidentally, “daytime” is the time except the sleep time of the test subject.

(2-3-1) Input of the Daytime Activity Relevant Values

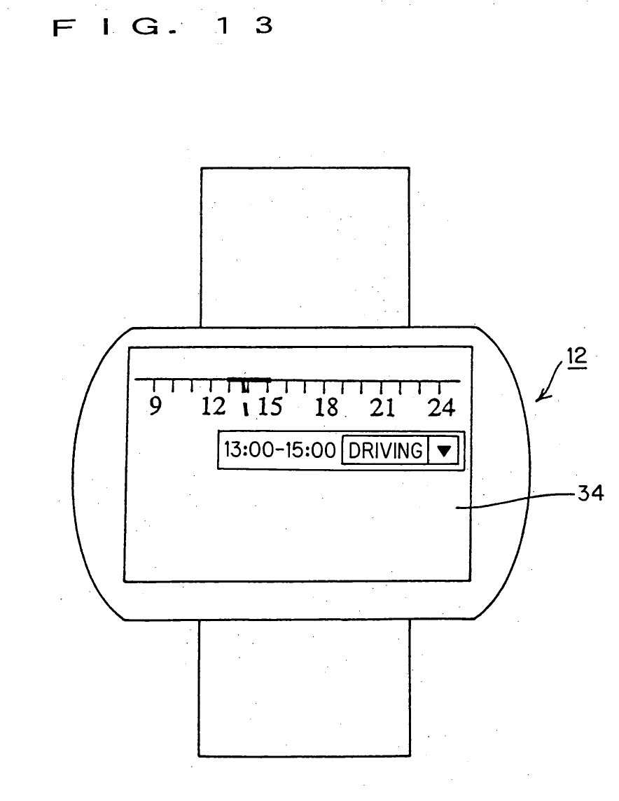

In the case of the input of the daytime activity relevant values, the respective items can be inputted in a selective manner or in an entry manner. FIG. 12 shows an input example of the selective input.

As shown in FIG. 12, an analog scale of each parameter is displayed, and each subjective value is inputted by a left and a right keys at both sides thereof. At this time, past maximum values of the test subject are regarded as the respective maximum values, and relative values thereto are inputted.

(2-3-2) Link to Schedule

Besides, the daytime activity relevant values are linked to data of a scheduler, and a judgment can be made based on the schedule data.

As shown in FIG. 13, with respect to the schedule data, when the range of a time zone in the time scale in which input is desired is specified, a pop-up window in which a schedule event corresponding thereto can be selected and inputted is opened. By this, the input can be made. Alternatively, a communication part (for example, Bluetooth module 42) to a PC, such as Bluetooth or USB, is incorporated, and data of schedule software of the PC may be transferred to the sensor.

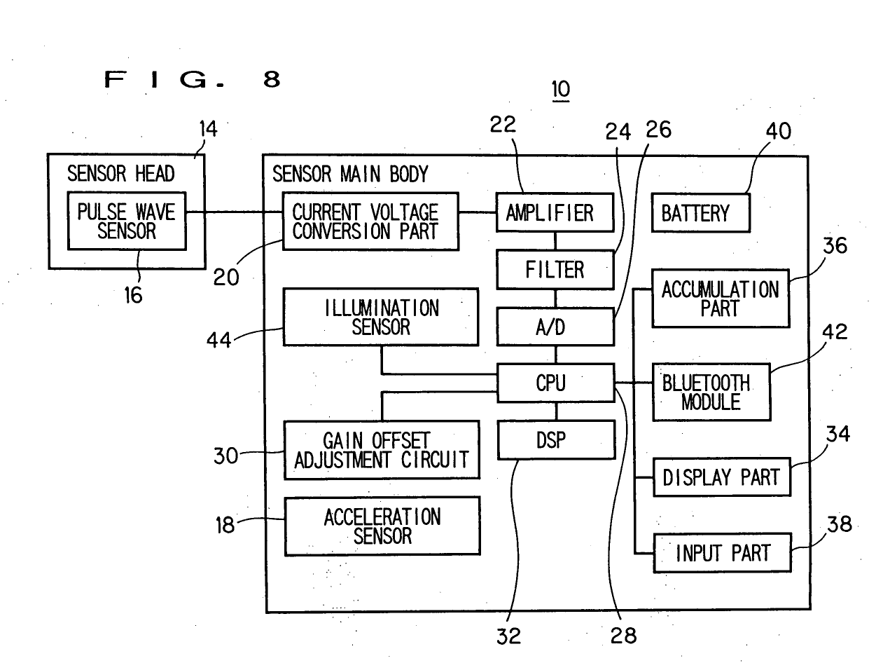

Standard values of the respective parameters corresponding to the events are previously set in a table as shown in FIG. 7, and the respective parameters are retrieved, acquired and determined correspondingly to the schedule event inputted as stated above.

(2-3-3) Measurement of Daytime Activity Relevant Values

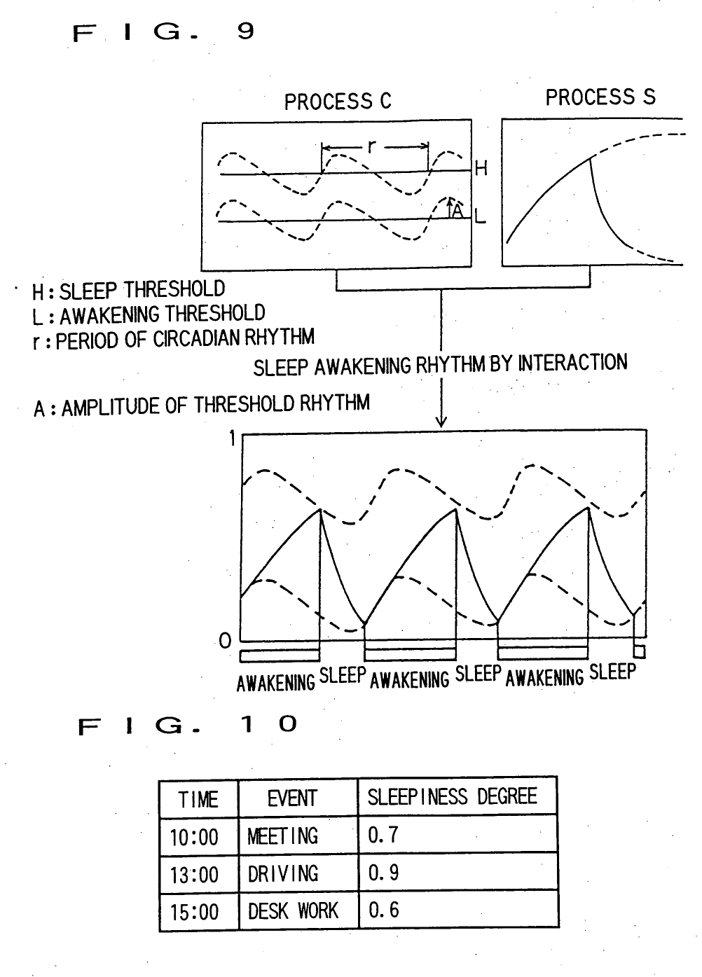

There is also a method for measuring the daytime activity relevant values. A sleepiness prediction apparatus 10 in the case where the measurement is made has a structure as shown in FIG. 8, and an illumination sensor 44 is added to the structure of FIG. 1.

The “daytime activity amount” is acquired from the acceleration. The integral value of scalar quantities, in one day, of variations obtained from the triaxial acceleration as described before is made the activity amount.

The “metabolic amount” is the integral value of pulses.

The “amount of exposure to light” is the integral value of illumination obtained by the illumination sensor 44.

The “stress value” is such that the above-mentioned autonomic nervous index values LF and HF are continuously measured also in the daytime, and the integral value of LF/HF is made the stress value. The integral value of HF may be acquired as the degree of relaxation.

(3) Calculation of Accumulated Sleepiness Degree S

The deep sleep time is made D, the REM sleep time is made R, the number of times REM sleep is made RT, and the total sleep time is made ST. The activity amount in the daytime is made A, the metabolic amount is made M, the amount of exposure to light is made L, and the stress value is made MS. By the weighted addition of all of these, an accumulated sleepiness degree S is calculated by an expression as described below. Here, S is the accumulated sleepiness degree on that day, SO is an accumulated sleepiness degree on the previous day, and C is a constant (offset). Incidentally, the accumulated sleepiness degree is the degree of sleepiness expected to be relieved or accumulated by the sleep history of the test subject and the daytime activity. Here, the sleepiness is defined to be the ratio (percentage) of the degree of subjective sleepiness felt by a person to the sleepiest state which is made 100%.
S=A1*A+A2*M+A3*L+A4*MS+C−(b1*D+b2*R+b3*RT+b4*ST)+SO
(4) Learning of Weight

As the constants A1, A2, A3, A4, b1, b2, b3, b4, and C, standard values for each age and gender are set, or learning is made with data in a learning period.

During the learning period, as the sleepiness degree S, a numerical value of 0 to 100% is inputted at any time in the daytime, and A, L and MS up to that time from the last input of the sleepiness degree are summed.

Besides, the sleepiness degree S is inputted also at the time of uprising, and at the same time, D, R, RT and ST are summed. From the data obtained by repeating this, the respective parameters are calculated by the least square method.

Incidentally, a deep sleep ratio is made D/ST, a REM sleep ratio is made R/ST, and an average period is made ST/RT, and these may be added as parameters.
S=A1*A+A2*M+A3*L+A4*MS+C−(b1*D+b2*R+b3*RT+b4*ST+b5*D/ST+b6*R/ST+b7*ST/RT)+SO

After the end of the learning period, the accumulated sleepiness degree accumulated up to that time can be calculated based on the measurement data and by the above expression.

(5) Biological Rhythm Sleepiness Degree K

It is known that the sleepiness varies not only by the accumulation and release as stated above, but also by the biological rhythm relating to a time, and a two-process model is proposed. The variation of the accumulation and release is explained as a process S, and the component varying according to the time is expressed as a process C as shown in FIG. 9. Thus, the sleepiness degree can be expressed by the combination of the accumulated sleepiness degree S due to the variation by the accumulation and release and the biological rhythm sleepiness degree K due to the time variation.

With respect to the biological rhythm sleepiness degree K, time-series data of standard sleepiness degree with respect to the easiness of occurrence of sleepiness is previously prepared in a table. The biological rhythm sleepiness degree K is expressed as follows as a function in order to detect the sleepiness degree corresponding to time t. This function retrieves the biological rhythm sleepiness degree K corresponding to the inputted time t from the table and displays it. In the case where a time not contained in the table is inputted, interpolation is made by data before and after that and a display is made.
K=k(t)

Alternatively, the biological rhythm sleepiness degree K is expressed by a sinusoidal composite function of a 24-hour circadian rhythm component and a 12-hour circasemidian rhythm component. Here, t denotes a time display of time, K1 and K2 denote weight coefficients of the respective circadian rhythm and circasemidian rhythm components, and θ1 and θ2 denote phases of the respective sine waves. With respect to K1, K2, θ1 and θ2, standard coefficients are set, or similarly to the above, for each test subject, learning is made from the change of the daytime sleepiness degree during the learning period.
K=K1*sin(2πt/24+θ1)+K2*sin(2πt/12+θ2)

The comprehensive sleepiness degree D is expressed as an average of the respective degrees of the accumulated sleepiness degree S and the biological rhythm sleepiness degree K by
D=(S+K)/2=(S+k(t))/2.

Alternatively, it may be expressed as the multiplication of the respective degrees by
D=S*K=S*k(t)
(6) Use Method of the Comprehensive Sleepiness Degree

Next, the use method of the comprehensive sleepiness degree will be described.

(6-1) First Use Method

As the first use method, there is a use method in which the sleepiness degree (excess or deficiency of sleep) at the present time, for example, at the time of uprising, is checked.

For example, when a “sleepiness check” button arranged on the screen is selected, the accumulated sleepiness degree is calculated in accordance with the above expression and by using the parameters measured up to that time, and is displayed together with the accumulated sleepiness degree before bedtime, so that the sleep state of the last night and the excess or deficiency of the sleep are displayed.

(6-2) Second Use Method

As the second use method, in addition to the prediction of the sleepiness at the present time point as stated above, there is also a case where future sleepiness is predicted. For example, sleepiness during future driving is predicted before the driving of an automobile, or sleepiness during future working hours is predicted before coming to work.

First, a relevant event or time is selected and inputted. In the case where the time is inputted, the event corresponding thereto is detected.

Next, the living state from the present time point to the predetermined event is inputted, or is acquired from the scheduler. With respect to each of the living states, a similar living state is acquired, and the daytime activity amount/metabolic amount (acceleration/pulse), the amount of exposure to light (illumination), and the stress value (integration of autonomic nervous balance) are acquired and calculated from input/measurement data at the time of the similar living state. Alternatively, from the table of the standard values as shown in FIG. 7, the living state is retrieved and the respective data is acquired.

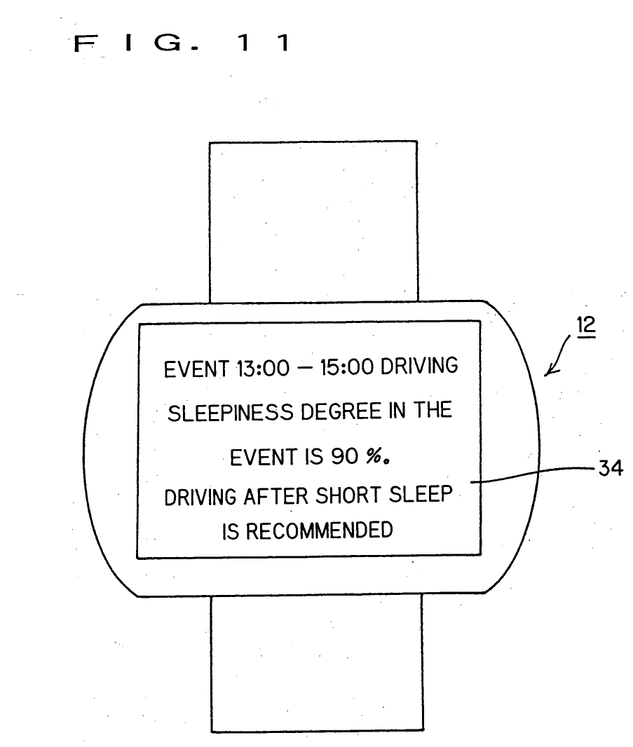

Based on this, the values of the respective parameters are substituted for the expression, and calculation is made with the time, so that the sleepiness can be predicted as shown in FIG. 10.

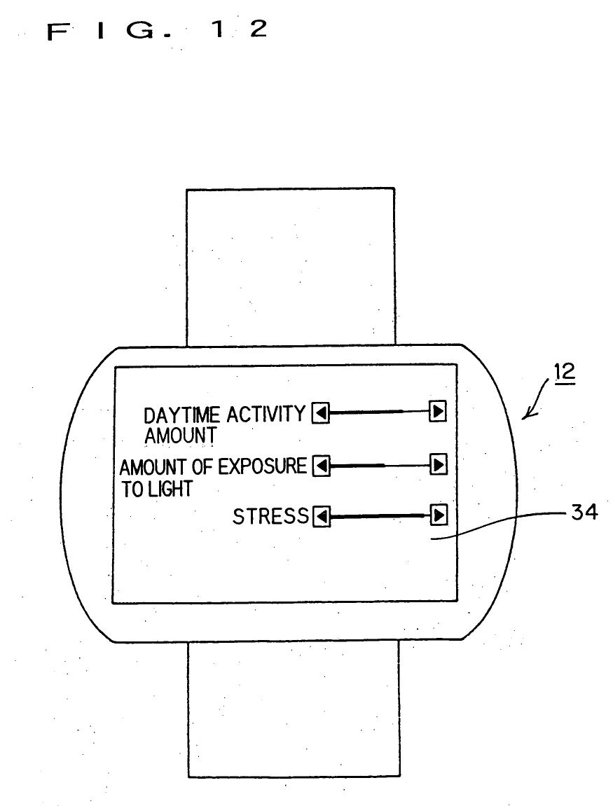

For example, in conjunction with the scheduler, the predicted sleepiness during an event, together with an alarm, is displayed before the event as shown in FIG. 11.

(6-3) Third Use Method

As the third use method, the sleepiness can also be used for the control of an alarm.

In order to provide an optimum sleep time on a holiday, at the time point of the set alarm time, in the case where the predicted sleepiness degree exceeds a set threshold, the alarm is driven.

When not exceeding (in the case of insufficient sleep), the alarm is extended to a predetermined time/next sleep cycle.

Besides, the alarm setting is not made, and when the sleepiness degree exceeds a set threshold, the alarm is driven, so that excessive sleep is prevented.

### MODIFIED EXAMPLE

The invention is not limited to the above respective embodiments, but can be modified variously within the scope not departing from the gist.

For example, in the embodiment, as shown in FIG. 2, although the pulse wave sensor 16 is of the head type in which it is mounted on the fingertip, this may be mounted on the palm of the hand by a sticking plaster, or may be such a form that the sensor is integrated with the main body 12 by using an infrared or red LED, and is mounted on the artery of the wrist.

What is claimed is:

1. A sleepiness prediction apparatus comprising:
a sleep state measurement processing part of measuring a sleep state relevant value relevant to a sleep state of a test subject;
a daytime activity acquisition part of inputting or measuring a daytime activity relevant value relevant to a daytime activity of the test subject; and
an accumulated sleepiness degree calculation processing part of calculating, based on the sleep state relevant value and the daytime activity relevant value, an accumulated sleepiness degree predicted to be accumulated by a sleep history of the test subject and the daytime activity.

2. A sleepiness prediction apparatus according to claim 1, further comprising:
a biological rhythm sleepiness degree calculation processing part of calculating a biological rhythm sleepiness degree based on a biological rhythm changing according to a time; and
a comprehensive sleepiness degree calculation processing part of calculating a comprehensive sleepiness degree corresponding to a time based on the accumulated sleepiness degree and the biological rhythm sleepiness degree.

3. A sleepiness prediction apparatus according to claim 1, wherein the sleep state measurement processing part includes:
an autonomic nervous index value measurement processing part of measuring an autonomic nervous index value of the test subject;
a body movement detection processing part of detecting a body movement state of the test subject;
an awakening/sleep judgment processing part of judging whether the test subject is wakeful or asleep based on the body movement state; and
a sleep state judgment processing part of obtaining a sleep state relevant value based on the autonomic nervous index value at a time when it is judged that the test subject is asleep.

4. A sleepiness prediction apparatus according to claim 1, wherein the sleep state relevant value is one of a deep sleep time of the test subject, a REM sleep time, the number of times of REM sleep, and a total sleep time.

5. A sleepiness prediction apparatus according to claim 1, wherein the daytime activity relevant value is one of a daytime activity amount of the test subject, a daytime metabolic amount of the test subject, an amount of exposure of the test subject to daytime light, and a stress value of the test subject.

6. A sleepiness prediction apparatus according to claim 5, wherein the daytime activity acquisition processing part includes:
an activity amount measurement processing part of measuring the daytime activity amount of the test subject;
a metabolic amount measurement processing part of measuring the daytime metabolic amount of the test subject;
a light irradiation amount measurement processing part of measuring the amount of the exposure of the test subject to the daytime light; and
a stress state measurement processing part of measuring the stress value relevant to stress of the test subject.

7. A sleepiness prediction apparatus according to claim 1, wherein the accumulated sleepiness degree calculation processing part multiplies a value of each of a deep sleep time of the test subject, a REM sleep time, the number of times of REM sleep, and a total sleep time, each of which is the sleep state relevant value, a daytime activity amount of the test subject, a daytime metabolic amount of the test subject, an amount of exposure of the test subject to daytime light, and a stress value of the test subject, each of which is the daytime action relevant value, by a weight and adds up them.

8. A sleepiness prediction apparatus according to claim 7, wherein the accumulated sleepiness degree calculation processing part includes:
a sleepiness degree input processing part by which the test subject inputs a sleepiness degree plural times; and
a weight learning processing part of calculating back to and learning the weight of each of the values by a least square method from the plural inputted sleepiness degrees, the respective values of the inputted or measured sleep state relevant values, and the respective values of the daytime activity relevant values.

9. A sleepiness prediction apparatus according to claim 2, wherein the biological rhythm sleepiness degree is expressed by a sinusoidal composite function of a 24-hour circadian rhythm component and a 12-hour circasemidian rhythm component.

10. A sleepiness prediction apparatus according to claim 2, further comprising a display processing part of displaying the accumulated sleepiness degree or the comprehensive sleepiness degree simultaneously with a schedule of the test subject.

11. A sleepiness prediction method, comprising the steps of:
measuring a sleep state relevant value relevant to a sleep state of a test subject;
inputting or measuring a daytime action relevant value relevant to a daytime activity of the test subject; and
calculating, based on the sleep state relevant value and the daytime activity relevant value, an accumulated sleepiness degree predicted to be accumulated by a sleep history of the test subject and a daytime activity.

12. A program of a sleepiness prediction method, causing a computer to realize:
a sleep state measurement function of measuring a sleep state relevant value relevant to a sleep state of a test subject;
a daytime activity acquisition function of inputting or measuring a daytime activity relevant value relevant to a daytime activity of the test subject; and
an accumulated sleepiness degree calculation function of calculating, based on the sleep state relevant value and the daytime activity relevant value, an accumulated sleepiness degree predicted to be accumulated by a sleep history of the test subject and a daytime activity.
